(19) United States
(12) Patent Application Publication
(10) Pub. No.: US 2007/0293935 A1
(43) Pub. Date: Dec. 20, 2007

(54) Stent deployment anchoring device

(75) Inventors: Kian G. Olsen, Bloomington, IN (US); Thomas A. Osborne, Bloomington, IN (US); Scott A. Todd, Bloomington, IN (US); Jason C. Urbanski, Ellettsville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/810,306

(22) Filed: Jun. 5, 2007

Related U.S. Application Data
(60) Provisional application No. 60/811,564, filed on Jun. 7, 2006.

(51) Int. Cl. A61F 2/84; A61M 29/02

(52) U.S. Cl. 623/1.12; 606/194

(57) ABSTRACT

An intraluminal stent delivery and deployment system and a method of delivering and deploying an intraluminal stent are described. The stent delivery and deployment system includes a catheter, an expandable stent disposed about a portion of the catheter, and at least one anchoring device attached to the catheter. The anchoring device has a proximal portion disposed distal of a midpoint of the stent. The anchoring device is deployed and engages a body vessel before the stent is expanded to a fully expanded configuration, thereby anchoring at least the catheter to the body vessel during the expansion of the stent.

### RELATED APPLICATIONS

The present patent document claims the benefit of the filing date under 35 U.S.C. §119(e) of Provisional U.S. Patent Application Ser. No. 60/811,564, filed on Jun. 7, 2006, which is hereby incorporated by reference.

### TECHNICAL FIELD

The present disclosure relates generally to medical devices and more specifically to deployment systems for expandable stents. BACKGROUND

Stents are useful in a variety of medical procedures and are often used to treat blockages, occlusions, narrowing ailments and other related problems that restrict flow through a passageway. Stents are typically designed as tubular support structures that are implanted within an artery or other vessel at a treatment site and then expanded from a compressed diameter to an expanded diameter. In the expanded state, the stent contacts and radially supports the inner wall of the passageway, thus preventing it from closing. Stents are generally classified as either balloon-expandable or self-expandable. Balloon-expandable stents expand in response to the inflation of a balloon. Self-expandable stents, on the other hand, expand automatically when released from a delivery device.

The delivery device for a self-expandable stent typically includes a catheter or inner carrier about which the stent is mounted in a compressed configuration, and an outer sheath surrounding the stent to maintain it in the compressed configuration for delivery into a body vessel. Once the delivery system is positioned at a treatment site within the vessel, the stent may be deployed to its expanded configuration by retracting the outer sheath with respect to the inner carrier. It is particularly desirable for the stent to be deployed in the proper location for effective treatment.

The deployment procedure is carried out by a skilled clinician who must manually manipulate components of the delivery system external to the body while monitoring, using x-ray fluoroscopy, the internal placement of the stent and the sheath. For example, to retract the sheath, the clinician holds an external hub connected to the inner carrier of the delivery system with one hand while moving a handle connected to the outer sheath using the other hand. The procedure must be carried out with great care and may prove difficult even for an experienced clinician. Even small unintended axial motions of the external hub may result in movements of the inner carrier that cause inaccurate placement of the stent within the vessel. Furthermore, the stent itself may “jump” in the distal direction upon final release from the sheath, a phenomenon that also may contribute to inaccurate positioning of the stent.

In light of these problems, it is apparent to the inventors that an improved deployment system would be desirable.

### BRIEF SUMMARY

A stent delivery and deployment system including at least one anchoring device that may allow for more accurate placement of an expandable stent within a body vessel compared to conventional deployment systems is described herein. The stent deployment system may help to minimize the impact of unintended axial movements by the clinician during expansion of the stent and may also mitigate the phenomenon of stent jumping as the stent is released from the outer sheath. Other advantages or uses of the system may also be possible.

The intraluminal stent deployment system includes a catheter, an expandable stent disposed about a portion of the catheter, and at least one anchoring device attached to the catheter. The anchoring device has a proximal portion disposed distal of a midpoint of the stent. The anchoring device deploys to contact a body vessel before the stent is expanded to a fully expanded configuration in the vessel.

A method of deploying an intraluminal stent includes providing a deployment assembly including a catheter, an expandable stent disposed about a portion of the catheter, and at least one anchoring device attached to the catheter. The method also provides for positioning the deployment assembly in a body vessel at a treatment site, and deploying the anchoring device to engage the body vessel, thereby anchoring the catheter to the body vessel. The method further provides for expanding the stent such that the stent reaches a fully expanded configuration after the anchoring device is deployed, and undeploying the anchoring device after the stent has reached the fully expanded configuration. Finally, the method provides for removing the catheter and the anchoring device from the body vessel, leaving the stent in the vessel in the fully expanded configuration.

### BRIEF DESCRIPTION OF THE DRAWINGS

FIGS. 1-1A show a stent delivery and deployment system, including external and internal (1A) components, according to one embodiment.

FIG. 2 shows internal components of a stent delivery and deployment system, according to one embodiment.

FIG. 3 shows internal components of a stent delivery and deployment system, according to a second embodiment.

FIG. 4 shows internal components of a stent delivery and deployment system, according to a third embodiment.

FIG. 5 shows internal components of a stent delivery and deployment system, according to a fourth embodiment.

FIG. 6 shows internal components of a stent delivery and deployment system, according to a fifth embodiment.

FIG. 7 shows internal components of a stent delivery and deployment system, according to a sixth embodiment.

FIG. 8 shows a perspective view of an anchoring device according to one embodiment.

FIGS. 9A-9C show views of a portion of the anchoring device of FIG. 8, according to three different embodiments.

FIGS. 10A-10D show an exemplary stent deployment procedure.

FIGS. 11A-11C show a second exemplary stent deployment procedure.

FIGS. 12A-12D show a third exemplary stent deployment procedure.

### DETAILED DESCRIPTION

Exemplary structures and elements having the same reference numbers in different figures may generally be assumed to be similar in structure and/or function.

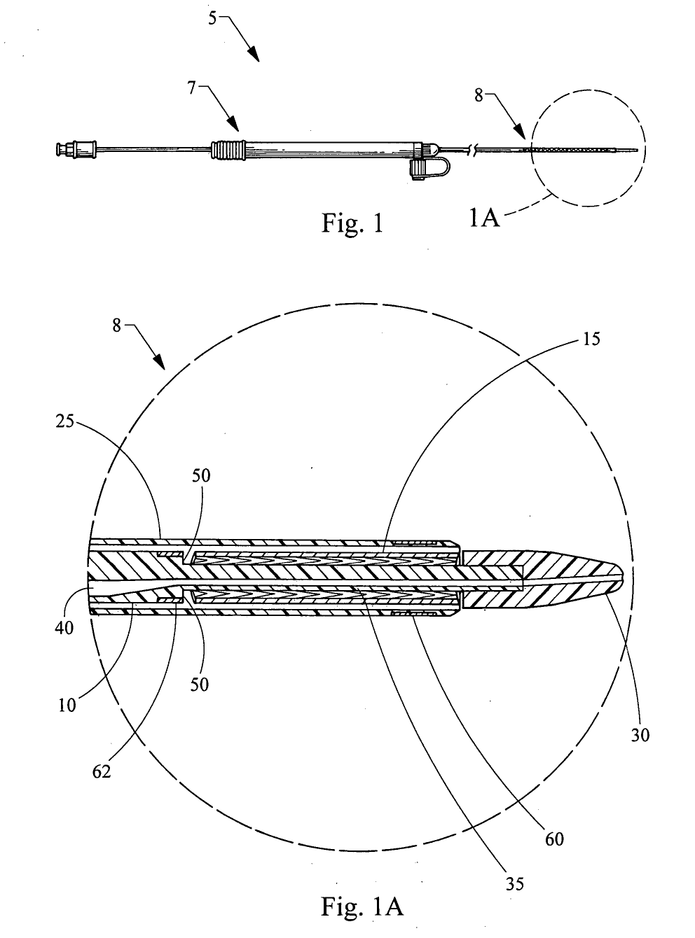

As shown in FIG. 1 and FIG. 1A, a stent delivery and deployment system 5 includes external components 7 that remain outside the body and internal components 8 that are inserted into a body vessel. Prior to deployment of an expandable stent 15 at a treatment site in the vessel, the internal components 8 of the deployment system 5 may include a catheter or inner carrier 10, a stent 15 disposed in an unexpanded configuration about a portion of the inner carrier 10, and at least one anchoring device (described below) in an undeployed configuration attached to the inner carrier 10. The deployment system 5 may also include an outer sheath 25 overlying the stent. In some embodiments, the outer sheath 25 may also overlie the undeployed anchoring device. The deployment system 5 may further include one or more radiopaque markers to aid in positioning the system 5 within the body vessel. Radiopaque markers may be disposed on the anchoring device, inner carrier 10, stent 15, and/or outer sheath 25, for example. FIG. 1A shows a first radiopaque marker 60 disposed on the outer sheath 25 and a second radiopaque marker 62 disposed on the inner carrier 10.

The inner carrier 10 may include a tapered tip region 30 at a distal end of the carrier 10 and a support region 35 disposed proximal to the tip region 30. In its compressed configuration, the stent 15 may be disposed about the support region 35 for delivery into the body vessel. The support region 35 may have a recessed outer diameter to accommodate the stent 10 without undesirably increasing the profile of the delivery system 5. The tip region 30 may be an integral part of the inner carrier 10, or it may be formed separately and then attached to the inner carrier 10. The inner carrier 10 may include a guidewire lumen 40 for passage over a guidewire during delivery of the stent 15 to a treatment site. The inner carrier 10 may also include one or more inflation lumens.

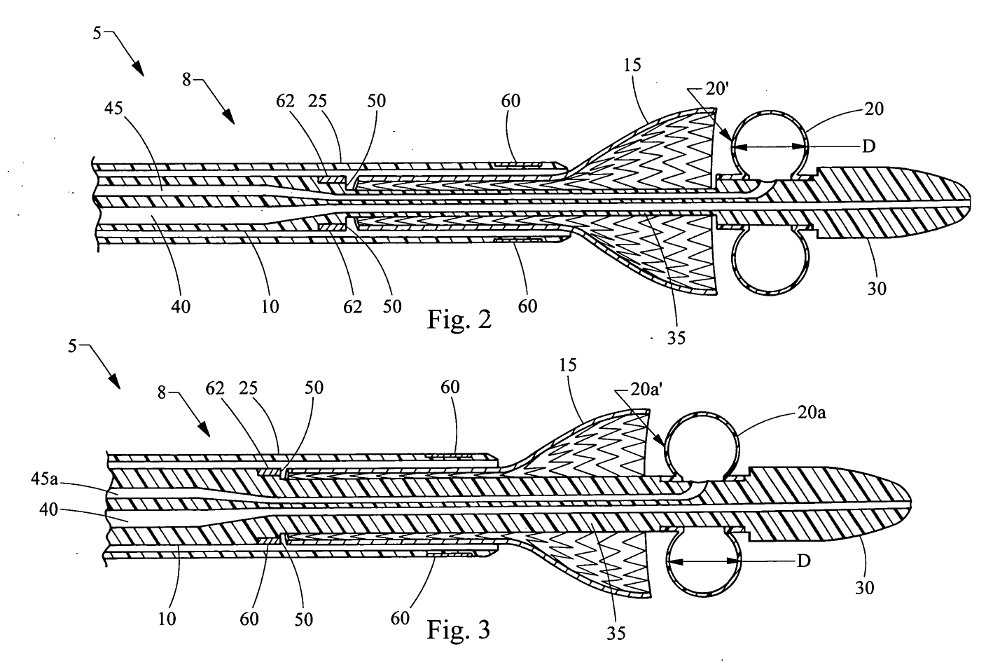

The tip region 30 and/or the support region 35 may accommodate the anchoring device, as is shown in FIGS. 2-7. According to some embodiments, the anchoring device may be deployed and come into contact with the body vessel before the stent 15 begins to expand so that the inner carrier 10 may be anchored to the body vessel during expansion of the stent 15. Preferably, the deploying of the anchoring device occurs before the stent 15 is fully expanded. In some embodiments, the anchoring device may anchor the stent 15, in addition to the inner carrier 10, to the body vessel during the expansion.

According to the embodiments shown in FIGS. 2-7, which are described in detail below, the stent 15 is self-expandable and is restrained by an outer sheath 25 overlying the stent 15 prior to the expansion. The figures show the stent 15 in a partially expanded configuration due to partial retraction of the outer sheath 25. The self-expandable stent 15 of the embodiments shown in FIGS. 2-7 reaches a fully expanded configuration when the outer sheath 25 is completely retracted and the stent 15 is released from the sheath 25. According to other embodiments of the stent delivery and deployment system 5, however, the stent may not be self-expandable. For example, the deployment system may be configured for delivering and deploying balloon expandable stents.

FIG. 2 is a schematic showing the internal components 8 of the stent deployment system 5 according to one embodiment. According to this embodiment, the anchoring device is an anchoring balloon 20 attached to the inner carrier 10 at a position distal of the stent 15. The anchoring balloon 20, which is shown in a deployed configuration, has a maximum axial dimension D which is less than an axial length of the stent 15. Preferably, the maximum axial dimension D of the anchoring balloon 20 may be less than one-half the axial length of the stent 15. The anchoring balloon 23 may be disposed in the tip region 30, as shown in FIG. 2. Preferably, a proximal portion 20′ of the anchoring balloon 20 is disposed distal of the stent 15. The outer sheath 25 may overlie the anchoring balloon 20 prior to deployment. In this case, the outer sheath 25 may be partly retracted before the anchoring balloon 20 is deployed.

In an alternative embodiment shown in FIG. 3, an anchoring balloon 20a may be disposed distal of the stent 15 in the support region 35. Consequently, the support region 35 may have a length sufficient to accommodate both the unexpanded stent 15 and the anchoring balloon 20a, which is shown in a deployed configuration. A proximal portion 20a′ of the anchoring balloon 20a is disposed distal of the stent 15. The outer sheath 25 may or may not overlie the anchoring balloon 20a prior to deployment, as desired. The anchoring balloon 20a has a maximum axial dimension D, which is less than an axial length of the stent 15. Preferably, the maximum axial dimension D of the anchoring balloon 20 is less than one-half the axial length of the stent 15.

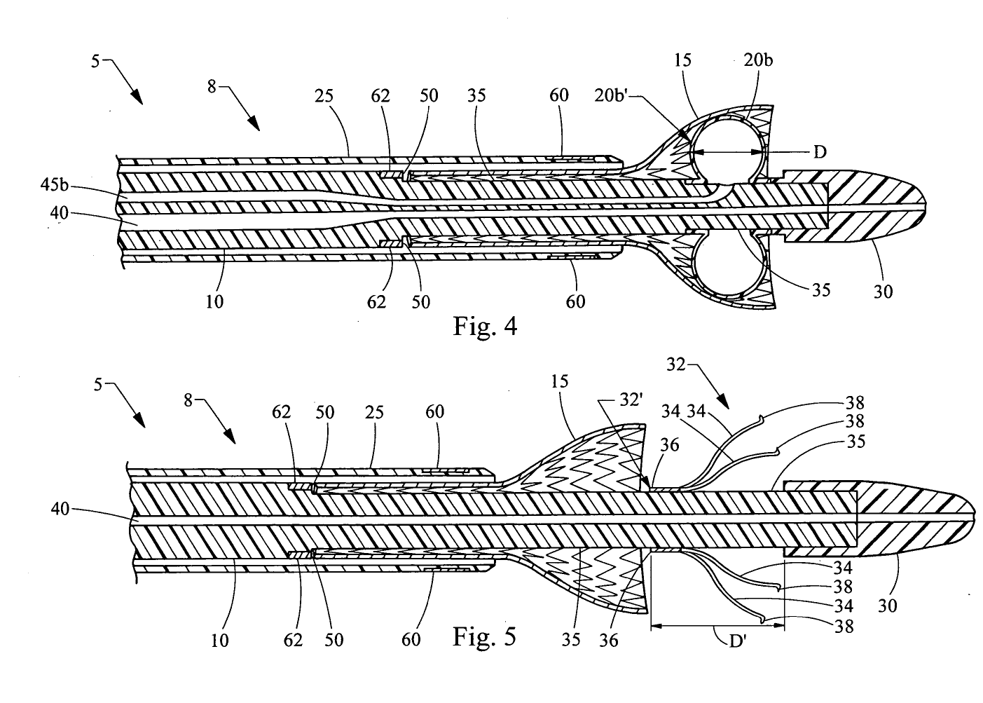

In another embodiment shown in FIG. 4, an anchoring balloon 20b may be disposed between the stent 15 and the portion of the inner carrier 10 about which the stent 15 is disposed. The anchoring balloon 20b is shown in a deployed configuration and has a maximum axial dimension D which is less than an axial length of the stent 15. Preferably, the maximum axial dimension D of the anchoring balloon 20b may be less than, one-half the axial length of the stent 15. A proximal portion 20b′ of the anchoring balloon 20b is disposed distal of a midpoint of the stent 15. The midpoint of the stent 15 is determined, for the purposes of the present disclosure, with respect to the axial length of the stent 15. According to this embodiment, the scent 15 and outer sheath 25 overlie the anchoring balloon 20b prior to deployment.

FIG. 5 is a schematic showing the internal components 8 of the stent deployment system 5 according to another embodiment. According to this embodiment, the anchoring device is a deployable arm device 32 including at least one arm 34 and a secured portion 36 which is secured to the inner carrier 10. The deployable arm device 32 is shown in a deployed configuration in FIG. 5. The device 32 has a maximum axial dimension D′ which is less than an axial length of the stent 15. Preferably, the maximum axial dimension D of the device 32 may be less than one-half the axial length of the stent 15.

The deployable arm device 32 may include two or more arms 34. As shown in FIG. 5, the deployable arm device 32 may include four arms 34. Alternatively, the deployable arm device 32 may include three, five, six, seven, or eight arms 34. The arms 34 may be disposed symmetrically about the circumference of the inner carrier 10. Alternatively, the arms 34 may be disposed asymmetrically about the circumference of the inner carrier 10. Each arm 34 may further comprise a projection or protrusion 38 for engaging the body vessel. The outer sheath 25 may overlie the deployable arm device 32 prior to deployment of the device 32, and the arms 34 on the device 32 may be spring-biased or otherwise designed to deploy automatically upon retraction of the sheath 25.

The deployable arm device 32 may be disposed in the support region 35 distal of the stent 15, as shown. Alternatively, the deployable arm device 32 may be disposed in another position distal of the stent 15, for example, in recesses that may be formed in the tip region 30 to accommodate the undeployed arms 34. Preferably, a proximal portion 32′ of the deployable arm device 32 is disposed distal of the stent 15.

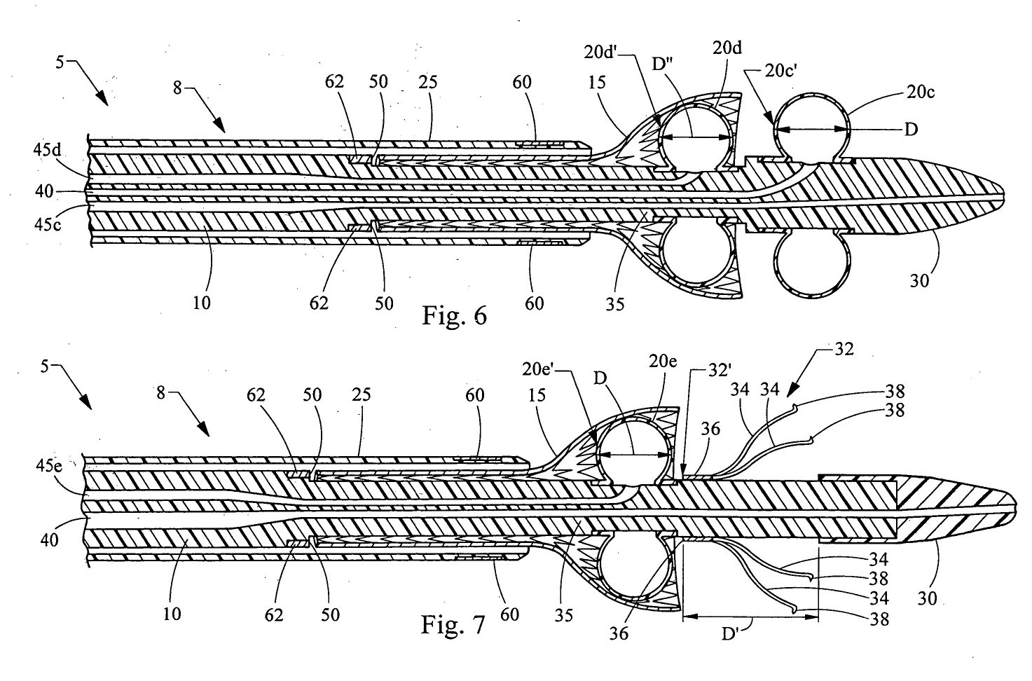

FIG. 6 is a schematic showing the internal components 8 of the stent deployment system 5 according to another embodiment in which two anchoring balloons are attached to the inner carrier 10. A first balloon 20c may be disposed at a position distal to the stent 15, and a second balloon 20d may be disposed at a position between the stent 15 and the portion of the inner carrier 10 about which the stent 15 is disposed. Each of the first balloon 20c and the second balloon 20d is shown in a deployed configuration, and each has a maximum axial dimension D, D″ which is less than an axial length of the stent 15. Preferably, the maximum axial dimension D, D″ is less than one-half the axial length of the stent 15. Preferably, a proximal portion 20c′ of the first balloon 20c may be disposed distal of the stent 15, and a proximal portion 20d′ of the second balloon 20d may be disposed distal of a midpoint of the stent 15. The midpoint of the stent 15 is determined, for the purposes of the present disclosure, with respect to the axial length of the stent 15. The first balloon 20c may be disposed, for example, in the tip region 30, as shown in FIG. 6. Alternatively, the first balloon 20c may be disposed in the support region 35 distal of the stent. In this case, the support region 35 may have a length sufficient to accommodate both the unexpanded stent 15 and the adjacent first balloon. The outer sheath 25 may overlie the second balloon 20d and the stent 15 prior to deployment of the stent 15 and the second balloon 20d. The outer sheath 25 may also overlie the first balloon 20c prior to deployment, if desired.

FIG. 7 is a schematic showing the internal components 8 of the stent deployment system 5 according to another embodiment. According to this embodiment, an anchoring balloon 20e and a deployable arm device 32 are attached to the inner carrier 10. Each of the anchoring balloon 20e and the deployable arm device 32 is shown in a deployed configuration, and each has a maximum axial dimension D, D′ which is less than an axial length of the stent 15. Preferably, the maximum axial dimension D, D′ is less than one-half the axial length of the stent 15.

The deployable arm device 32 is shown in a deployed configuration in FIG. 7. The deployable arm device 32 may include at least one arm 34 and a secured portion 36 which is secured to the inner carrier 10. Each arm 34 may further comprise a projection or protrusion 38 for engaging the body vessel. Preferably, the deployable arm device 32 may include two or more arms 34. The deployable arm device 32 may include, for example, four arms 34, as shown in the schematic. Alternatively, the deployable arm device 32 may include three, five, six, seven, or eight arms 34. The arms 34 may be disposed symmetrically about the circumference of the inner carrier 10. Alternatively, the arms 34 may be disposed asymmetrically about the circumference of the inner carrier 10. The outer sheath 25 may overlie the deployable arm device 32 prior to deployment of the device 32. The arms 34 on the device 32 may be spring-biased or otherwise designed to deploy automatically upon retraction of the sheath 25. The outer sheath 25 also overlies the anchoring balloon 20e (and the stent) prior to deployment.

As shown in FIG. 7, the anchoring balloon 20e is disposed between the stent and the portion of the inner carrier 10 about which the stent is disposed, and the deployable arm device is disposed in the support region 35 distal of the stent 15. The support region 35 may have a length sufficient to accommodate both the stent 15 in an unexpanded configuration and the deployable arm device 32 prior to deployment. The deployable arm device 32 may alternatively be disposed in the tip region 30, for example, in recesses formed to accommodate the undeployed arms 34. Preferably, a proximal portion 32′ of the deployable arm device 32 is disposed distal of the stent 15, and a proximal portion 20e′ of the anchoring balloon 20e is disposed distal of a midpoint of the stent 15. For the purposes of this disclosure, the midpoint of the stent is determined with respect to an axial length of the stent 15.

Anchoring balloons suitable for the stent delivery systems described herein may be made of one or more polymers, including, for example, polyethylene teraphthalate (PET), polyethylene, nylon, or thermoplastic elastomers (e.g., a block copolymer of polyether glycol and polybutylene terephthalate (PBT)). The thickness of the polymer may be within the range of from about 0.0005 inch to about 0.02 inch (from about 0.013 mm to about 0.5 mm) to provide an anchoring balloon that may exert a sufficient force when deployed to remain anchored in place, but which is sufficiently compact in an uninflated state for delivery into the vessel. Anchoring balloons suitable for the stent delivery systems described herein may be formed using medical device balloon fabrication methods known in the art, such as those described in U.S. Pat. No. 6,488,653, which is assigned to Wilson-Cook Medical Inc. and is hereby incorporated by reference.

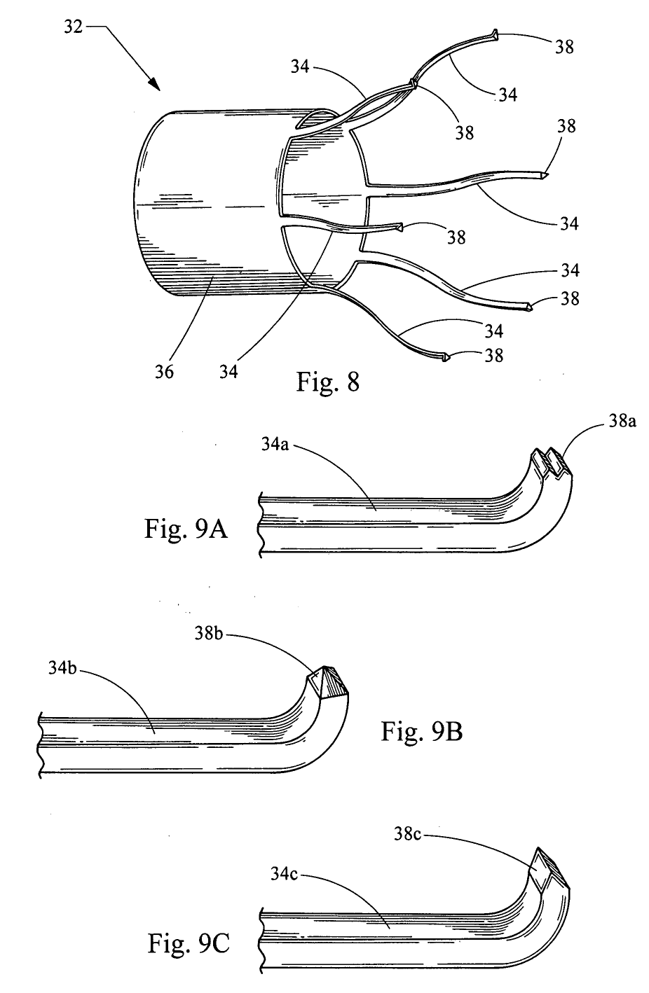

One embodiment of the deployable arm device 32 which comprises at least one arm 34 and a secured portion 36 is shown in perspective view in FIG. 8. The device may be formed of one or more biocompatible materials. Preferably, the deployable arm device is made of a biocompatible metal or metal alloy, such as, for example, stainless steel, nickel-titanium (e.g., Nitinol), or Conichrome (an alloy of cobalt, nickel, chromium and other elements)., According to one embodiment, the deployable arm device may be formed from a superelastic or shape-memory material.

Preferably, the secured portion 36 of the deployable arm device 32 is a tubular structure having an inner diameter sized to fit over a portion of the inner carrier. The secured portion 36 may be slid over the inner carrier from the distal end, according to one embodiment, and then secured to the inner carrier using a biocompatible adhesive. Alternatively, the secured portion 36 may be secured to the inner carrier using any other attachment means known in the art, such as, for example, a friction fit, threads, or barbs. To facilitate the attachment of the secured portion 36 of the deployable arm device 32 to the inner carrier, the tip region of the inner carrier may be formed separately and assembled with the inner carrier only after the secured portion 36 is disposed in place.

The one or more arms 34 of the deployable arm device 36 may be integrally formed with the secured portion. For example, the deployable arm device 32, including the secured portion 36 and the one or more arms 34, may be fabricated from a single tube by laser cutting. If desired, the end of each arm may include a projection or protrusion 38 for anchoring into the vessel. Such projections may produced by forming and grinding methods known in the art. The projections 38 may have any of the exemplary geometries shown in FIGS. 9A-9C, or an alternative structure which is similarly effective for engaging the vessel wall. Preferably, the arms 34 on the device 32 may be spring-biased or otherwise designed to deploy automatically upon retraction of a sheath overlying the device 32.

The self-expandable stents described herein are preferably made of a superelastic or shape memory material. The term “superelastic material,” as used herein, refers to a material that exhibits a substantial amount of elastic (i.e., recoverable) deformation, or strain, in response to an applied stress. Typically, such materials can achieve elastic strains of at least several percent. In the case of self-expandable stents made from superelastic materials, stress may be applied to deform the stent into a compressed configuration for delivery within a sheath into a body vessel. Once the stent is placed at a treatment site within the vessel, the outer sheath may be retracted to release the stress, thereby allowing the stent to expand to its original, undeformed configuration. According to one embodiment, the superelastic material used to form the described self-expandable stents 15 includes nickel and titanium. The superelastic material may be a nickel-titanium alloy, such as Nitinol. The nickel-titanium alloy may also include a ternary element, a quaternary element and/or additional elements. An example of a commercially available stent formed of a superelastic nickel-titanium alloy is the Zilver® stent, manufactured by Cook, Inc. (Bloomington, Ind.).

The inner carrier or catheter may be made of one or more polymers, such as, for example, a polyamide (e.g., nylon), fluorocarbon (e.g., polytetrafluoroethylene (PTFE)), polyether block amide (PEBA), polyolefin, or polyimide. The catheter may further include, embedded within the one or more polymers, a metallic (e.g., stainless steel) reinforcement structure to impart kink resistance and column strength to the catheter. Conventional catheter manufacturing methods known in the art, including, for example, extrusion, bonding and/or molding, may be employed to fabricate the catheter.

The catheter may include one or more lumens, including, for example, a guidewire lumen, and one or more inflation lumens if needed to inflate the anchoring device(s) attached to the catheter. The guidewire lumen may extend from the proximal end to the distal end of the catheter, in the case of a long-wire catheter, or from a point intermediate between the distal and proximal ends to the distal end, in the case of a short-wire or rapid-exchange catheter.

Outer sheaths known in the art for use with self-expandable stent deployment systems may be used in embodiments of the present stent deployment system. Typically, the outer sheath may be made of one or more polymers, such as, for example, nylon or PTFE, and may also include a metallic reinforcement structure, such as an embedded coil disposed in a helical configuration, for kink resistance and column strength.

Radiopaque markers may be attached to various components of the deployment system, including, for example, the outer sheath, the stent, the inner core, and the anchoring device. These markers may be made of a radiopaque material, that is, a material that strongly absorbs x-ray radiation and is thus readily visible using an x-ray imaging device, such as a fluoroscope. Preferably, the radiopaque material is also biocompatible. The radiopaque material may include, for example, gold, iridium, niobium, palladium, platinum, silver, tantalum, tungsten, or an alloy thereof, such as platinum-iridium.

Descriptions of several representative procedures for using the stent delivery and deployment system 5 are set forth below. Exemplary external and internal components 7, 8 of the stent delivery and deployment system are shown; other configurations may also be possible. According to these examples, the stent 15 is self-expandable. During the procedures, the clinician may observe the impact of his or her external maneuverings on the intraluminal environment by using x-ray fluoroscopy, which illuminates radiopaque markers attached to components of the deployment system.

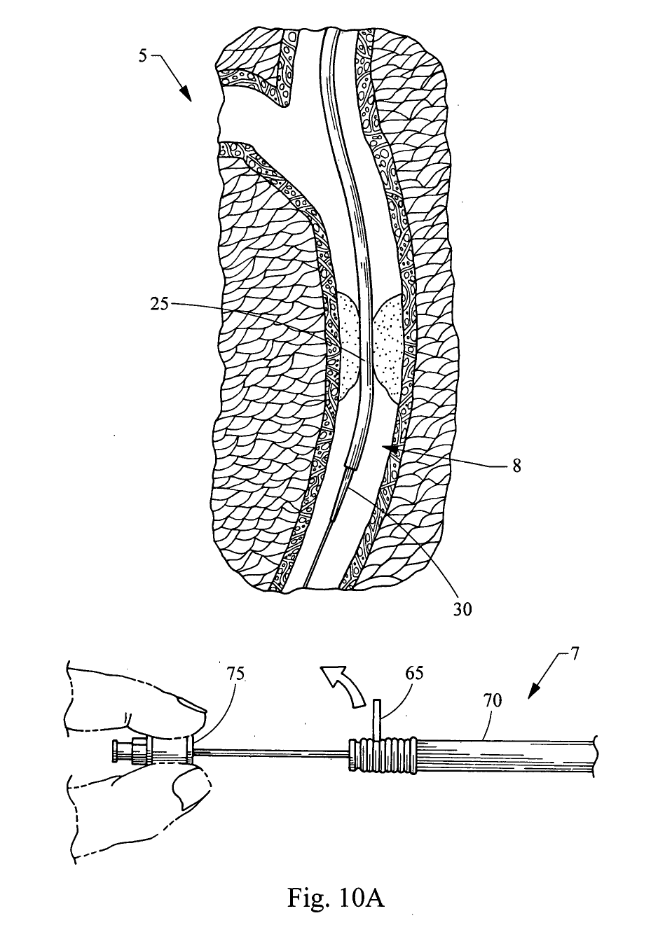

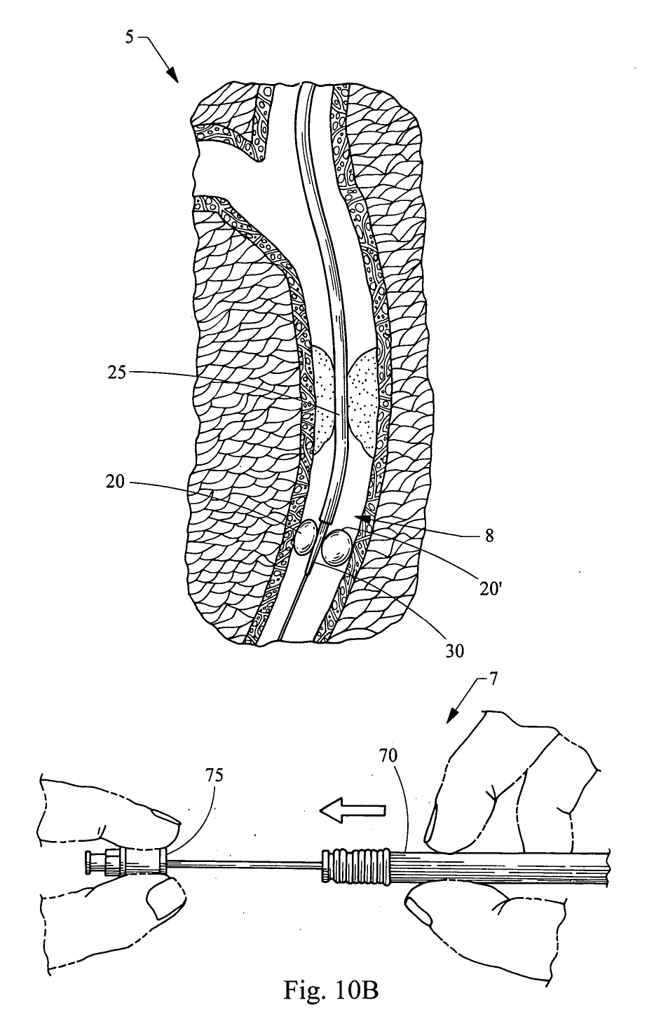

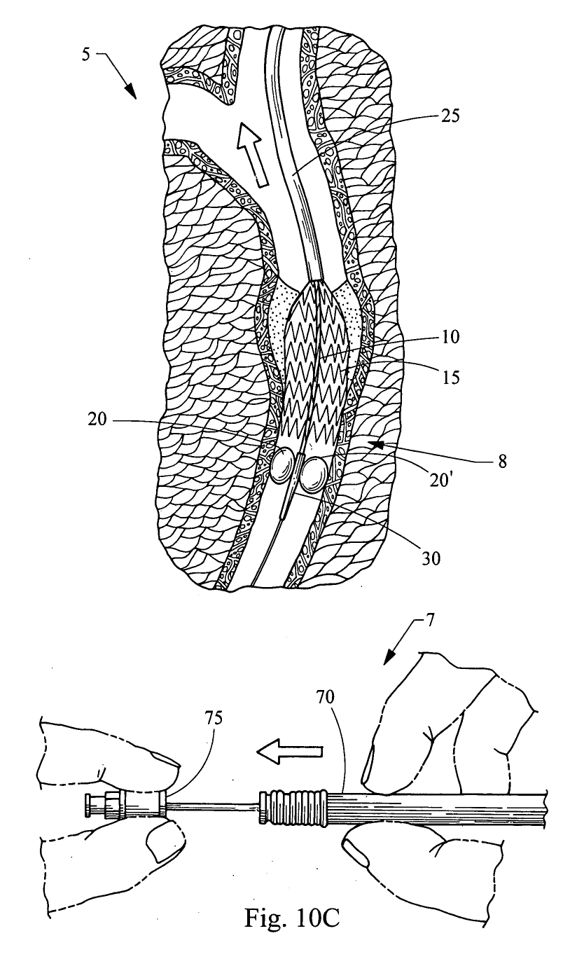

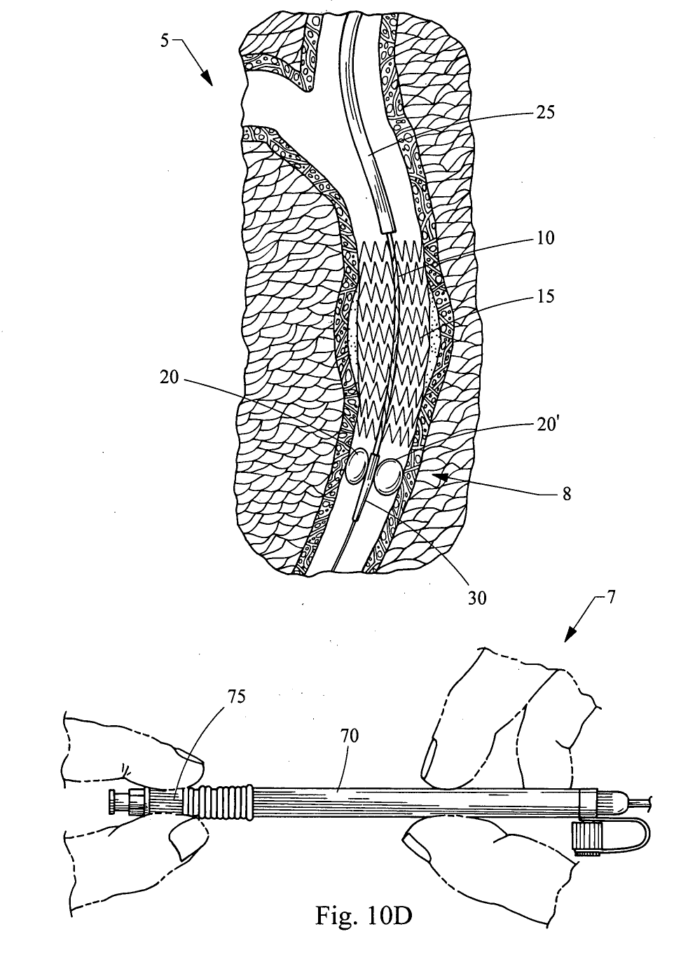

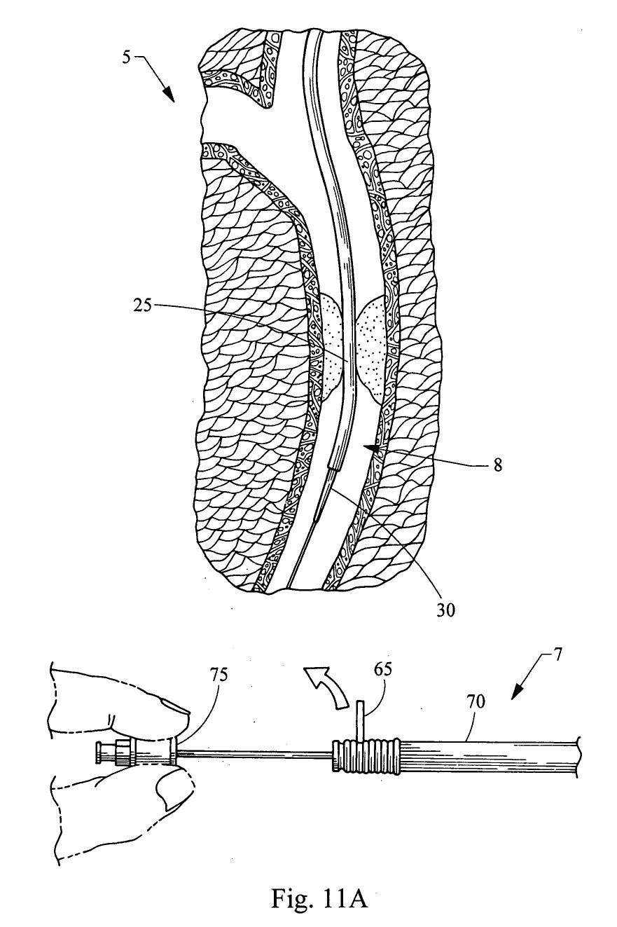

FIGS. 10A-10D show an exemplary procedure for using the stent deployment system 5 with views of both the external and internal components 7, 8 of the delivery and deployment system 5. According to this example, a single anchoring device (e.g., an anchoring balloon 20) is attached to the inner carrier 10 at a position distal of the stent 15. A proximal portion 20′ of the anchoring balloon 20 is disposed distal of the stent 15.

In FIG. 10A, the internal components 8 of the deployment system 5 are positioned at a treatment site in a body vessel, and the clinician prepares to deploy the uninflated anchoring balloon 20 and the compressed stent 15. An outer sheath 25 overlies the anchoring balloon 20 and the stent 15, according to this embodiment. Externally, the clinician removes a safety lock 65, thereby disengaging a handle 70 attached to the outer sheath 25 from a hub 75 connected to the inner carrier 10.

In FIG. 10B, the clinician begins to retract the outer sheath 25 in a proximal direction by manual manipulation of the handle 70 external to the body. As the sheath 25 is retracted, the undeployed anchoring balloon 20 is exposed. According to this embodiment, the anchoring balloon 20 is disposed at a position in the tip region 30. To inflate the anchoring balloon 20, an inflation fluid such as, for example, a saline solution or a radiopaque solution (e.g., 50% by volume of a contrast medium and 50% by volume of saline solution), may be pumped through an inflation lumen 45 (shown for example in FIG. 2) using inflation techniques known in the art. Once the anchoring balloon 20 is inflated and in contact with the body vessel, the inner carrier 10 may be anchored in place at the treatment site.

In FIG. 10C, the clinician further retracts the outer sheath 25 by manual manipulation of the handle 70 external to the body. As the outer sheath 25 is retracted, the proximal end of the stent 15 comes into contact with a ledge or step 50 (visible in FIGS. 1-7) located proximally of the stent 15 on the inner carrier 10. The ledge 50 restrains the stent 15 from movement in the proximal direction as the outer sheath 25 is further retracted. As the stent 15 is exposed during retraction of the sheath 25, the stent 15 expands gradually, beginning with its distal end.

As shown in FIG. 10D, the clinician fully retracts the outer sheath 25 by moving the external handle 70 to contact the hub 75, and the stent 15 is completely released from the sheath 25. After the stent 15 is fully deployed in the vessel and is no longer in contact with the outer sheath 25, the anchoring balloon 20 may be deflated by removing inflation fluid through the inflation lumen 45 (see FIG. 2). The inner carrier 10, outer sheath 25, and anchoring balloon 20 may then be removed from the vessel, leaving the stent 15 in an expanded configuration at the treatment site.

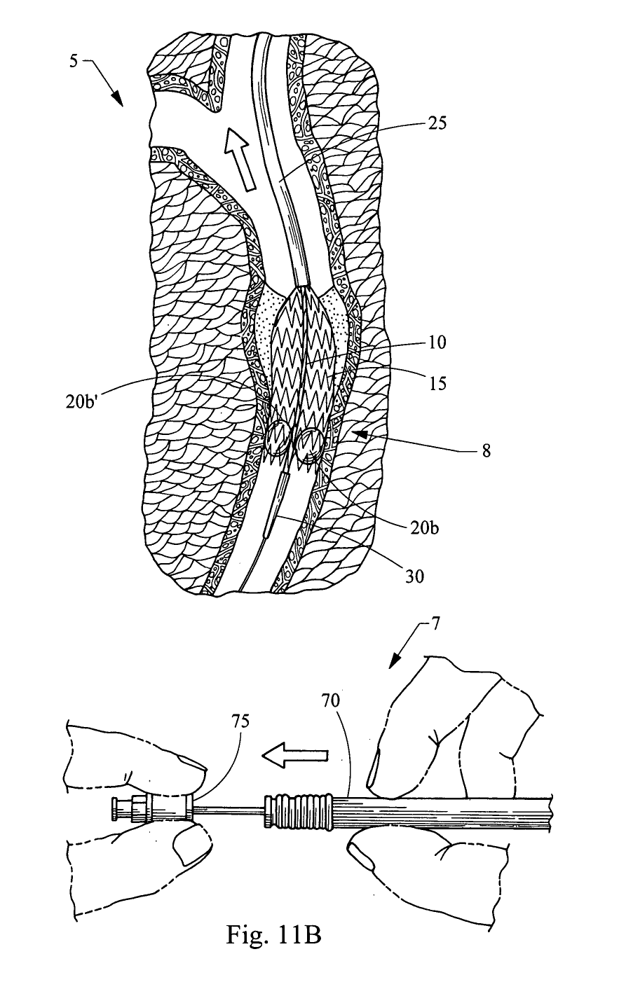

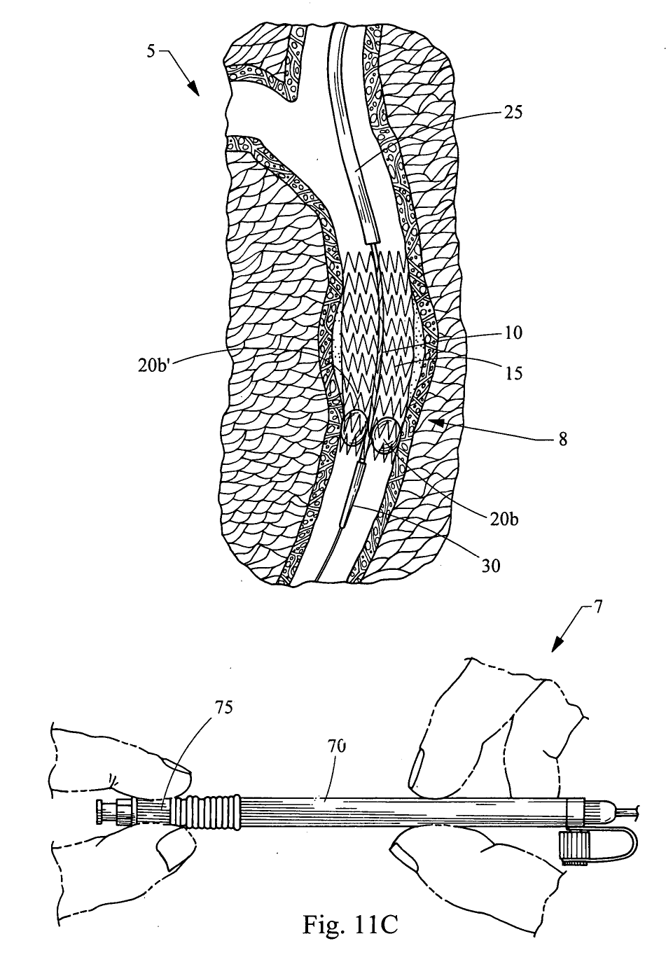

FIGS. 11A-11C show a second exemplary procedure for using the stent delivery and deployment system 5 with views of both the external and internal components 7, 8 of the deployment system 5. According to this example, a single anchoring device (e.g., an anchoring balloon 20b) is attached to the inner carrier 10 at a position between the stent 15 and the portion of the inner carrier 10 about which the stent 15 is disposed. The stent 15 and an outer sheath 25 overlie the undeployed anchoring balloon 20b. The anchoring balloon 20b is positioned near the distal end of the stent 15, so that deployment of the anchoring balloon 20b may be achieved before the stent 15 is fully expanded. A proximal portion 20b′ of the anchoring balloon 20b is disposed distal of the midpoint of the stent 15.

In FIG. 11A, the internal components 8 of the deployment system 5 are positioned at the treatment site. The clinician prepares to deploy the anchoring balloon 20b and the stent 15, each of which is in an undeployed configuration within the outer sheath 25. External to the body, the clinician removes a safety lock 65, thereby disengaging the handle 70 (connected to the outer sheath 25) from the hub 75 (connected to the inner core 10).

In FIG. 11B, the clinician begins to retract the outer sheath 25 by manual manipulation of the handle 70 external to the body. As the outer sheath 25 is retracted, the proximal end of the stent 15 comes into contact with a ledge or step 50 (visible in FIGS. 1-7) located proximally of the stent 15 on the inner carrier 10. The ledge 50 restrains the stent 15 from movement in the proximal direction as the outer sheath 25 is further retracted. The stent 15 gradually expands during retraction of the sheath 25, beginning with its distal end. As the clinician is retracting the sheath 25, inflation fluid may be pumped through the inflation lumen 45b (shown in FIG. 4) to inflate the anchoring balloon 20b using inflation techniques known in the art. The anchoring balloon 20b may be deployed and may anchor both the inner carrier 10 and the expanding stent 15 to the body vessel prior to complete expansion of the stent 15.

As shown in FIG. 11C, the clinician fully retracts the outer sheath 25 by moving the external handle 70 to contact the hub 75, and the stent 15 is completely released from the sheath 25. After the stent 15 is fully deployed in the vessel and is no longer in contact with the outer sheath 25, the anchoring balloon 20b may be deflated by removing inflation fluid through the inflation lumen 45b (see FIG. 4). Then, the inner carrier 10, outer sheath 25, and anchoring balloon 20b may be removed from the vessel, leaving the stent 15 in an expanded configuration at the treatment site.

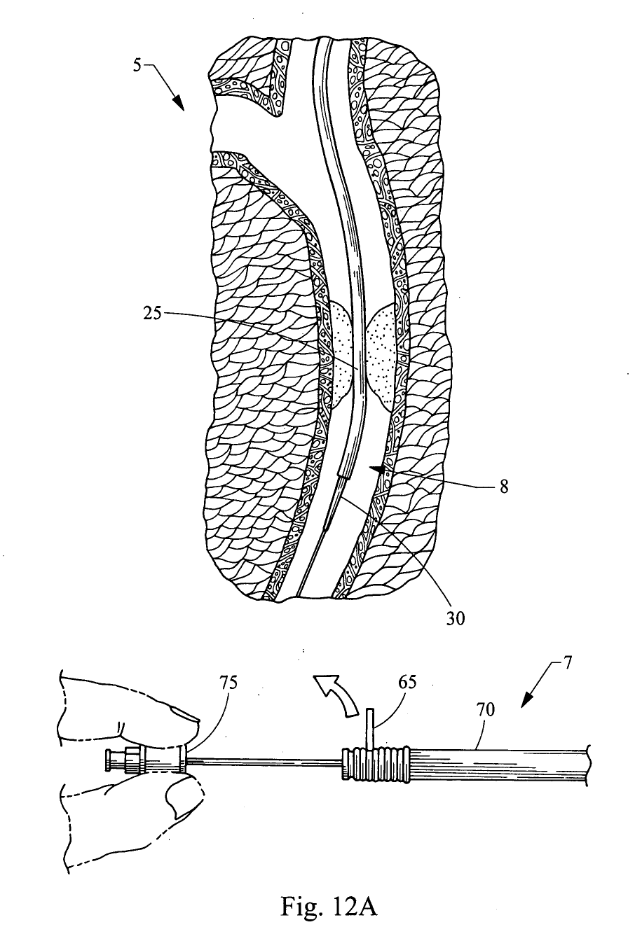

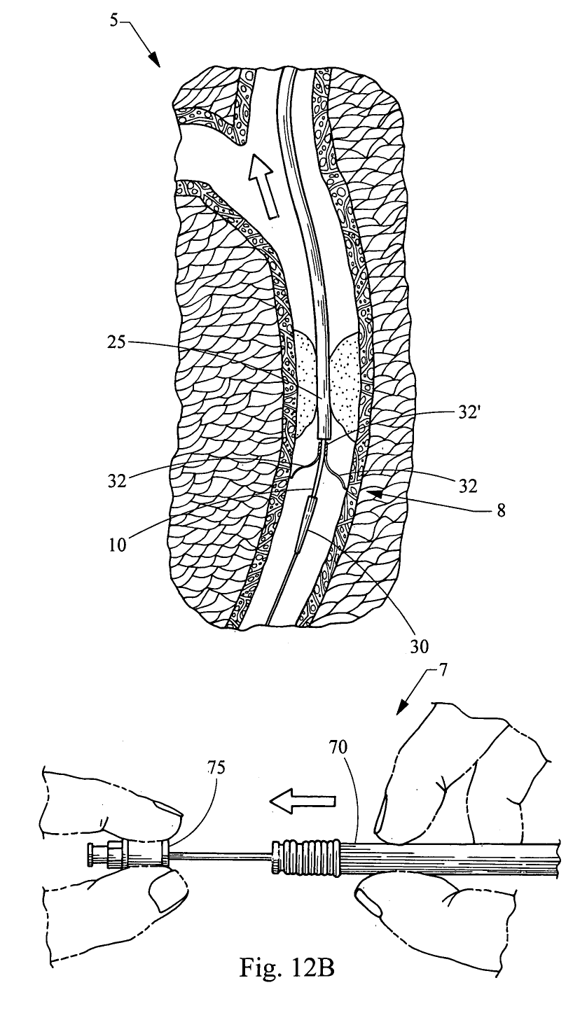

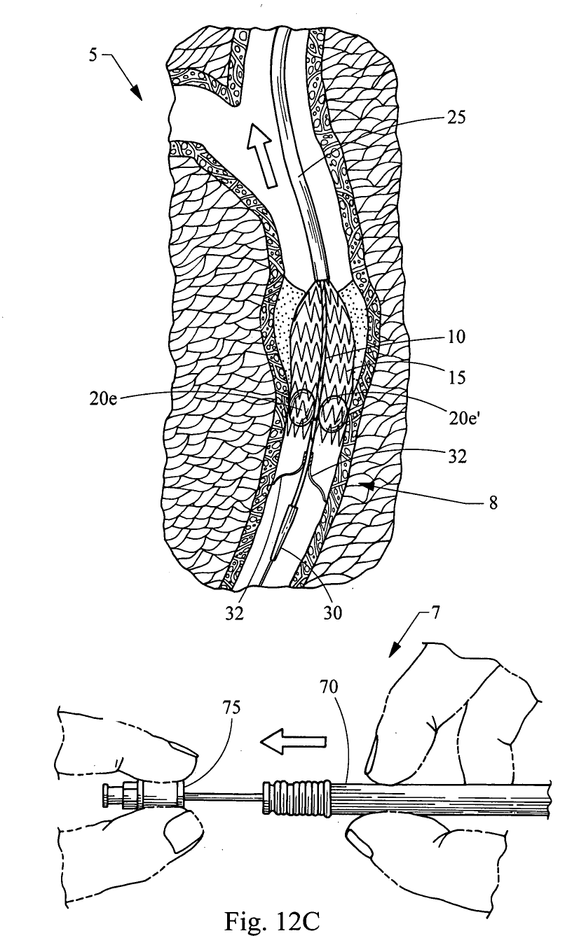

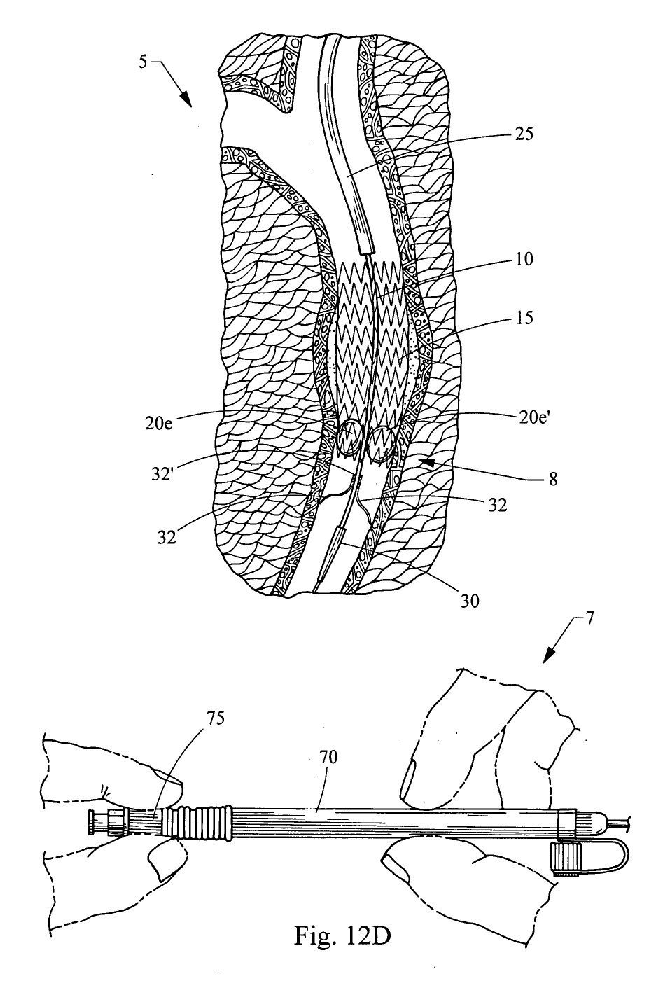

FIGS. 12A-12D show a third exemplary procedure for using the stent delivery and deployment system 5 with views of both the external and internal components 7, 8 of the deployment system 5. According to this embodiment, the stent 15 is self-expandable and two anchoring devices (e.g., an anchoring balloon 20e and a deployable arm device 32) are attached to the inner carrier 10. The deployable arm device 32 is disposed at a position distal to the stent 15, and the anchoring balloon 20e is disposed at a position between the self-expandable stent 15 and the portion of the inner carrier 10 about which the stent 15 is disposed. Further, a proximal portion 32′ of the deployable arm device 32 is disposed distal of the stent 15, and a proximal portion 20e′ of the anchoring balloon 20e is disposed distal of a midpoint of the stent 15. The midpoint of the stent is determined with respect to an axial length of the stent 15. The anchoring balloon 20e is positioned near the distal end of the stent 15 so that deployment of the anchoring balloon 24 may be achieved before the stent 15 is completely expanded. The stent 15 and the outer sheath 25 overlie the undeployed anchoring devices 20e, 32, of this example.

In FIG. 12A, the internal components 8 of the deployment system 5 are positioned at the treatment site in a body vessel. The clinician prepares to deploy the more distally positioned anchoring device, the deployable arm device 32, which is in an undeployed configuration within the outer sheath 25. External to the body, the clinician removes a safety lock 65, thereby disengaging the handle 70 (connected to the outer sheath 25) from the hub 75 (connected to the inner core 10).

In FIG. 12B, the clinician partially retracts the outer sheath 25 by manual manipulation of the handle external to the body, and the arms 34 of the deployable arm device 32 extend to contact the vessel, anchoring the inner carrier 10 in place at the treatment site.

In FIG. 12C, the clinician further retracts the outer sheath 25 by moving the handle 70 external to the body. As the outer sheath 25 is retracted, the proximal end of the stent 15 comes into contact with a ledge or step 50 (visible in FIGS. 1-7) located proximally of the stent 15 on the inner carrier 10. The ledge 50 restrains the stent 15 from movement in the proximal direction as the outer sheath 25 is further retracted. The stent 15 gradually expands during retraction of the sheath 25, beginning with its distal end. As the clinician is retracting the sheath 25, inflation fluid may be pumped through an inflation lumen 45e (shown in FIG. 7) to inflate the anchoring balloon 20e using inflation techniques known in the art. The anchoring balloon 20e may be deployed and anchor both the inner carrier 10 and the expanding stent 15 to the body vessel prior to complete expansion of the stent 15.

As shown in FIG. 12D, the clinician fully retracts the outer sheath 25 by moving the external handle 70 to contact the hub 75, and the stent 15 is completely released from the sheath 25. After the stent 15 is fully deployed in the vessel and is no longer in contact with the outer sheath 25, the anchoring balloon 20e may be deflated by removing inflation fluid through the inflation lumen 45e (see FIG. 7), and the outer sheath 25 may be moved in a distal direction to collapse the deployable arm device 32 into an undeployed configuration. Then, the inner carrier 10, outer sheath 25, anchoring balloon 20e, and deployable arm device 32 may be removed from the vessel, leaving the stent 15 in an expanded configuration at the treatment site.

The accuracy of stent placement at a treatment site within a vessel may be improved by using the stent delivery and deployment system described herein. One or more anchoring devices may be used to anchor the inner carrier and, in some embodiments, the stent itself to the vessel during expansion of the stent. The stent deployment system may help to minimize the impact of unintended axial movements by the clinician during expansion of the stent, and may further mitigate or eliminate the phenomenon of stent jumping as the stent is released from the outer sheath.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein.

Furthermore, the advantages described above are not necessarily the only advantages of the invention, and it is not necessarily expected that all of the described advantages will be achieved with every embodiment of the invention.

We claim:

1. An intraluminal stent deployment system, comprising:
a catheter;
an expandable stent disposed about a portion of the catheter;
at least one anchoring device attached to the catheter and having a proximal portion disposed distal of a midpoint of the stent,
wherein the anchoring device deploys to contact a body vessel before the stent is expanded to a fully expanded configuration in the vessel.

2. The stent deployment system according to claim 1, wherein the proximal portion of the anchoring device is disposed distal of the stent.

3. The stent deployment system according to claim 1, wherein a maximum axial dimension of the anchoring device is smaller than an axial length of the stent.

4. The stent deployment system according to claim 3, wherein the maximum axial dimension of the anchoring device is smaller than one-half of the axial length of the stent.

5. The stent deployment system according to claim 1, wherein the anchoring device is disposed between the stent and the portion of the catheter about which the stent is disposed.

6. The stent deployment system according to claim 1, wherein the stent is self-expandable and further comprises an outer sheath overlying the stent, the outer sheath being retractable to expand the stent.

7. The stent deployment system according to claim 6, wherein the outer sheath overlies both the stent and the anchoring device.

8. The stent deployment system according to claim 1, wherein the anchoring device is a balloon.

9. The stent deployment system according to claim 1, wherein the anchoring device comprises a deployable arm device comprising at least one arm and a secured portion secured to the catheter.

10. The stent deployment system according to claim 9, wherein the secured portion comprises a tubular structure disposed about a region of the catheter, and wherein the arm is integrally formed with the tubular structure.

11. The stent deployment system according to claim 1, comprising first and second anchoring devices, the first anchoring device having a proximal portion disposed distal to the stent and the second anchoring device being disposed between the stent and the portion of the catheter about which the stent is disposed.

12. The intraluminal stent deployment system according to claim 11, wherein the first anchoring device is one of a first balloon and a deployable arm device comprising at least one arm and a secured portion secured to the catheter, and wherein the second anchoring device is a second balloon.

13. A method of deploying an intraluminal stent, comprising:
providing a deployment assembly comprising:
a catheter;
an expandable stent disposed about a portion of the catheter;
at least one anchoring device attached to the catheter;
positioning the deployment assembly in a body vessel at a treatment site;
deploying the anchoring device to engage the body vessel, thereby anchoring the catheter to the body vessel;
expanding the stent, the stent reaching a fully expanded configuration after the deploying of the anchoring device;
undeploying the anchoring device after the stent has reached the fully expanded configuration; and
removing the catheter and the anchoring device from the body vessel, leaving the stent in the vessel in the fully expanded configuration.

14. The method according to claim 13, wherein the deploying of the anchoring device comprises retracting a sheath overlying the anchoring device, and wherein undeploying the anchoring device comprising advancing a sheath over the anchoring device.

15. The method according to claim 13, wherein the deploying of the anchoring device comprises passing an inflation fluid through an inflation lumen of the catheter, and wherein undeploying the anchoring device comprises removing the inflation fluid through the inflation lumen.

16. The method according to claim 13, wherein the deploying of the anchoring device occurs before the stent begins to expand, the anchoring device being disposed distal of the stent.

17. The method according to claim 13, wherein the deploying of the anchoring device engages the stent with the body vessel, thereby anchoring the stent to the body vessel before the stent reaches the fully expanded configuration.

18. The method according to claim 13, comprising deploying a first anchoring device and deploying a second anchoring device, wherein deploying the second anchoring device engages the stent with the body vessel, thereby anchoring the stent to the body vessel before the stent reaches the fully expanded configuration.

19. The method according to claim 18, wherein deploying at least one of the first anchoring device and the second anchoring device comprises passing an inflation fluid through an inflation lumen of the catheter, and wherein deploying the second anchoring device and expanding the stent comprises retracting a sheath overlying at least the second anchoring device and the stent.

20. The method according to claim 19, wherein the sheath overlies the first anchoring device and deploying the first anchoring device comprises retracting the sheath, and wherein undeploying the first anchoring device and the second anchoring device comprises at least one of removing inflation fluid through an inflation lumen and advancing the sheath over the first anchoring device.
